(19) United States
(12) Patent Application Publication
(10) Pub. No.: US 2002/0098184 A1
(43) Pub. Date: Jul. 25, 2002

(54) T-cell dependent vaccine and the cell surface receptor CD28

(76) Inventor: Andrew William Heath, Sheffield (GB)

Correspondence Address: KLAUBER & JACKSON, 411 HACKENSACK AVENUE, HACKENSACK, NJ, 07601

(21) Appl. No.: 10/072,583

(22) Filed: Feb. 8, 2002

Related U.S. Application Data
(63) Continuation of application No. 09/171,063, filed on Oct. 9, 1998.

(30) Foreign Application Priority Data
Apr. 13, 1996 (GB) 9607711.0

(51) Int. Cl. A61K 39/395; A61K 39/00

(52) U.S. Cl. 424/141.1; 424/184.1

(57) ABSTRACT

The invention relates to a novel vaccine suitable for enhancing T-cell dependent immunity comprising a T-cell dependent antigen, such as a soluble protein, and an associated adjuvant, the adjuvant being adapted to stimulate a T-cell lymphocyte via the cell surface receptor CD28.

The invention relates to a method of manufacture and a system for the production of a novel T-cell dependent vaccine; and also the vaccine thereof.

It is known that the immune system works on the basis of recognition and thus the ability to distinguish between self and non-self. Recognition of non-self, or invading material, is followed by a sequence of steps that are designed to kill or eliminate the non-self material. As knowledge of the immune system grows and molecular biological techniques advance it has become possible to advantageously manipulate the various steps in an immune response in order to enhance the nature of that response. Thus, for example, it has become possible to manufacture a wide range of vaccines using recombinant material and thus manufacture a range of vaccines which were not previously available either because the relevant material was not obtainable or had not before been produced.

The specific immune system is made up of lymphocytes which are able to recognise specific antigens. B lymphocytes recognise antigens in their native conformation through surface immunoglobulin receptors, and T lymphocytes recognise protein antigens that are presented as peptides along with self molecules known as MHC, on the surface of antigen presenting cells. There are a variety of antigen presenting cells including B lymphocytes. T lymphocytes may be further subdivided into cytotoxic T lymphocytes, which are able to kill virally infected “target” cells, and T helper lymphocytes. T “helper” lymphocytes are able to help B lymphocytes to produce specific antibody, or to help macrophages to kill intracellular pathogens.

Although T-cells recognise antigen bound to MHC molecules through structures on their surface known as the “T-cell receptor” (TCR), recognition of antigen through TCR results only in a partial activation of the T-cell. T lymphocytes require a second signal, in addition to antigen recognition, in order to become fully activated. One of the most important of these so called second signals, is delivered through a molecule recognised by the monoclonal antibody CD28. In the subsequent description the cell surface molecule will hereinafter be referred to as CD28. Binding of CD28 to one of its ligands on an antigen presenting cell is said to result in “co-stimulation” of the T lymphocyte. There are two known natural ligands for CD28, known as B7.1 and B7.2, and binding of either of these ligands to CD28, in conjunction with stimulation through the T-cell receptor, results in “co-stimulation” of the T-cells, and leads to enhanced T-cell proliferation and cytokine secretion. There is some evidence that binding of B7.1 or B7.2 to CD28 results in qualitative differences in signalling, and may result in pushing the T lymphocyte towards differentiation into a T helper 1 type cell (B7.1) or a T helper 2 type cell (B7.2). T helper 1 cells are potent macrophage activating cells, while T helper 2 type cells are good enhancers of some antibody responses.

It follows from the above that the immune system is highly complex and involves the interaction, typically in synchronised fashion, of a number of molecules in order to provide a given response.

Briefly, vaccines are therapeutic materials which are derived, either directly or indirectly from non-self material and, typically, either attenuated so as to reduce the associated virulence or diluted so as to reduce the associated virulence prior to administration to humans or other animals so as to stimulate the immune system. Moreover, since the immune system is provided with memory, this initial stimulation is able to protect against future infection with pathogens comprising the vaccine material.

With the investigation of new vaccines it has been realised that the effectiveness of vaccines can be enhanced by the co-administration of an adjuvant. Briefly, an adjuvant is an agent which assists or enhances the effectiveness of an antigen or vaccine.

Our investigations have led us to produce a novel vaccine which essentially involves the co-administration of antigen and adjuvant and more specifically our investigations have led us to use a murine model of CD28 activation of T-lymphocytes. This involves the co-administering of an antigen physically associated with an antibody to murine CD28 and referred to as anti-CD28.

More specifically, our investigations have lead us to produce a T-cell dependent vaccine, and thus a vaccine which is characterised by the above immune response, involving a T-cell dependent antigen and an agent that is able to bind to the T lymphocyte surface receptor CD28.

We believe that one of the reasons our vaccine is so effective is because it manipulates the action of CD28 in a T-cell dependent immune response. In this respect it is noted that CD28 is constitutively expressed and therefore, amongst other things, the timing of the administration of the vaccine is not crucial, A further advantage of our vaccine is that it provides a safe immunological adjuvant to antigens that are soluble proteins. This has hitherto been a problem and there has been a pressing need to provide such adjuvants for soluble proteins.

It is therefore an object of the invention to provide a novel vaccine for use in T-cell dependent immunity.

It is a further object of the invention to provide a safe immunological adjuvant for use in such a vaccine and also for use in enhancing the immune response to soluble proteins.

It is further object of the invention to provide a method for the production of a vaccine of the invention.

It is a further object of the invention to provide a system for the production of the vaccine of the invention.

According to a first aspect of the invention there is therefore provided a vaccine suitable for enhancing T-cell dependent immunity comprising a T-cell dependent antigen, or part thereof, and an associated adjuvant which is adapted to stimulate a T lymphocyte surface receptor, CD28.

Reference herein to the term vaccine is intended to include a wide variety of vaccines including, but not limited to, contraceptive vaccines, immunotherapy vaccines and prophylactic or therapeutic vaccines.

In a preferred embodiment of the invention said antigen is soluble and ideally a protein.

Ideally stimulation of said CD28 is via binding of said adjuvant, or a part thereof, to at least a part of CD28.

In a preferred embodiment of the invention said antigen and adjuvant are bound or crosslinked theretogether.

More preferably said adjuvant is an antibody, either polyclonal or monoclonal, but ideally monoclonal, which is adapted to bind to said CD28.

More preferably still, said adjuvant is a humanised monoclonal antibody, which is adapted to bind to said CD28.

In a preferred aspect of the invention said antibody may be whole or, alternatively, comprise only those domains which are effective at binding CD28, and in particular selected parts of CD28.

In another embodiment of the invention, said adjuvant is one of the natural ligands for CD28, B7.1 or B7.2 ideally produced as a recombinant protein, or a CD28 binding portion of B7.1 or B7.2; or indeed any other ligand, or part thereof, that binds CD28 or part thereof.

In a further embodiment, the CD28 ligand may not be a naturally occurring CD28 ligand but represent an agent that due to its biochemical characteristics has an affinity for CD28.

The finding that B7.1 and B7.2 binding of CD28 may differentially enhance TH1 versus TH2 type T-cell responses means that the adjuvant can be selected so as to influence the nature of the T-cell response. This can be done by selecting the nature of the ligand i.e. B7.1 or B7.2 or by selecting antibodies that bind to different sites of CD28.

In its broadest context, reference herein to the term adjuvant includes reference to any string of amino acids or ligand which is selected so as to bind to at least a part of CD28.

In a preferred aspect the recombinant vaccine antigen, and the adjuvant will be produced as a chimeric fusion protein.

It will be apparent to those skilled in the art that said antigen may be any T-cell dependent antigen and thus any antigen which is capable of eliciting a T-cell dependent response.

Ideally said antigen and/or adjuvant is in the form of an immunostimulating complex, or liposomes or biodegradable microspheres, so favouring a cytotoxic T-cell response which is ideally enhanced.

Alternatively said vaccine comprises an emulsion of the antigen and adjuvant ideally in oil.

According to a second aspect of the invention there is provided an adjuvant for enhancing an immune response to soluble protein wherein said adjuvant comprises an agent adapted to stimulate a T lymphocyte surface receptor, CD28.

Preferably said stimulation of said CD28 is via binding of said adjuvant, or a part thereof, thereto.

Ideally, said adjuvant is an antibody, either polyclonal or monoclonal, but ideally monoclonal, which is adapted to bind to said CD28.

In a preferred aspect of the invention said antibody may be whole or, alternatively, comprise only those domains which are effective at binding CD28, and in particular selected parts of CD28.

In this aspect of the invention said adjuvant is coadministered with said soluble protein and ideally soluble protein that is effective at eliciting a T-cell dependent immune response.

More preferably further still said adjuvant is cojoined to said protein.

According to a yet further aspect of the invention there is provided a method for the manufacture of a novel vaccine capable of eliciting a T-cell dependent immune response which method comprises the selection of a suitable T-cell dependent antigen, or part thereof, and combination of said antigen with an adjuvant wherein said adjuvant is adapted to stimulate a T lymphocyte receptor, CD28.

In yet a further preferred method of the invention said antigen is recombinantly manufactured and/or said adjuvant is recombinantly manufactured.

In yet a further preferred embodiment of the method of the invention said antigen and adjuvant are bound or crosslinked theretogether, or produced as a recombinant chimeric fusion protein.

Preferably said stimulation of said CD28 is via binding of said adjuvant, or a part thereof, thereto.

Ideally, said adjuvant is an antibody, either polyclonal or monoclonal, but ideally monoclonal, which is adapted to bind to said CD28.

In a preferred aspect of the invention said antibody may be whole or, alternatively, comprise only those domains which are effective at binding CD28, and in particular selected parts of CD28

According to a yet further aspect of the invention there is provided a system for the manufacture of a vaccine capable of eliciting a T-cell dependent immune response which system comprises a cell expressing a selected T-cell dependent antigen, or part thereof, and also an adjuvant capable of stimulating a T lymphocyte receptor CD28.

More preferably still both said antigen and said adjuvant are adapted so as to be secreted from said cell. This may be undertaken by providing both the antigen and adjuvant with secretion signals or providing for the production of a single piece of material comprising both the antigen and the adjuvant and having a single secretion signal associated therewith. It will be evident that in the former instance the said antigen and adjuvant will be found in associated but unbound or uncrosslinked manner in the supernatant of the system, and in the latter instance said antigen and adjuvant will be cojoined in the supernatant of the system.

Preferably said stimulation of said CD28 is via binding of said adjuvant, or a part thereof, thereto.

Ideally, said adjuvant is an antibody, either polyclonal or monoclonal, but ideally monoclonal, which is adapted to bind to said CD28.

In a preferred aspect of the invention said antibody may be whole or, alternatively, comprise only those domains which are effective at binding CD28, and in particular selected parts of CD28.

According to a yet further aspect of the invention there is provided an isolated DNA molecule encoding either or both said antigen and/or said adjuvant of the invention.

We are currently unsure as to the precise way in which our vaccine works but, without limitation to the scope of the invention, we speculate that a fraction of the vaccine becomes bound to the B lymphocyte and because it comprises T-cell dependent antigen it becomes internalised, processed and a fraction thereof is presented on the B lymphocyte cell surface so as to form a complex which the T lymphocyte binds in usual manner. A further fraction of the vaccine is bound to be B lymphocyte prior to internalisation and processing. Thus as B lymphocytes have concurrently bound on their cell surface processed T-cell dependent antigen/MHC Class II complex and also T-cell dependent antigen/anti-CD28 we speculate that in this circumstance antigen specific T-cells might receive a stronger, earlier signal than would normally be the case, with a high degree of cross-linking of CD28 occurring prior to any natural B7.1 or B7.2 expression by the B cell.

However, our hypothesis remains to be tested and is therefore simply presented for the reader's edification rather than as an explanation of the working of the invention.

The invention will now be described by way of example only with reference to the following figures, but it is of note that in the examples avidin has been used as the soluble antigen because it binds with high affinity to biotinylated proteins, thus producing cross-linking, wherein;

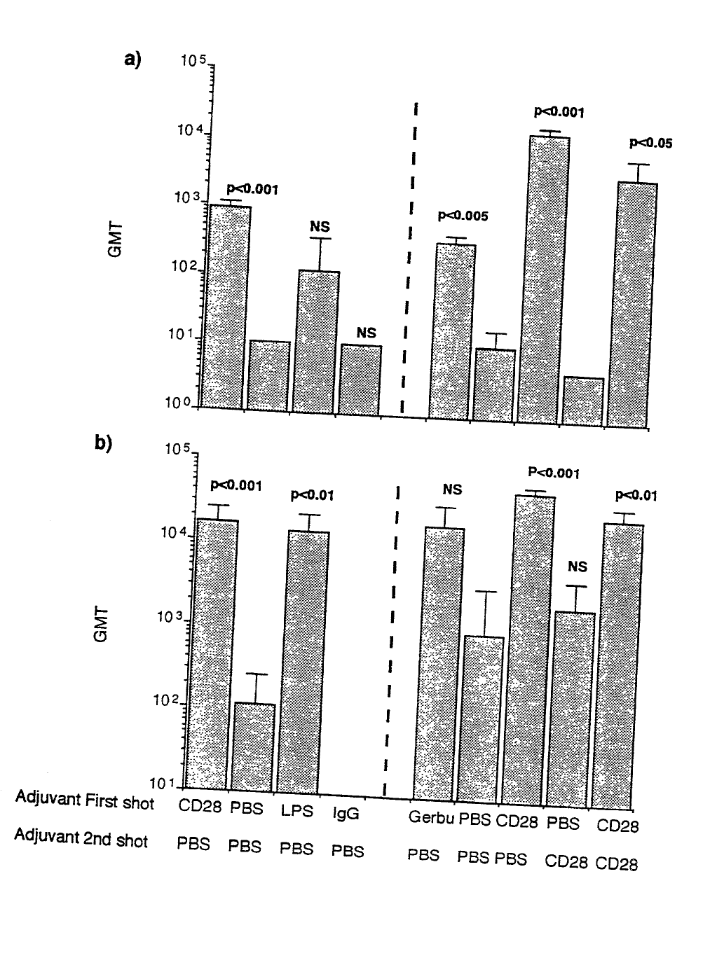

FIG. 1—The y-axis shows the geometric means of endpoint titrations against avidin, by ELISA, following primary and secondary immunisations with 10 μg avidin, plus 10 μg “adjuvant” or no adjuvant (PBS). The inclusion of biotinylated anti-CD28 significantly enhances both the primary anti-avidin antibody response, and the secondary response to avidin alone. IgG represents a biotinylated control hamster IgG (anti-CD28 is a hamster IgG antibody). LPS is Salmonella Typhosa lipopolysaccharide, at 10 μg per mouse and Gerbu represents a commercially available adjuvant.

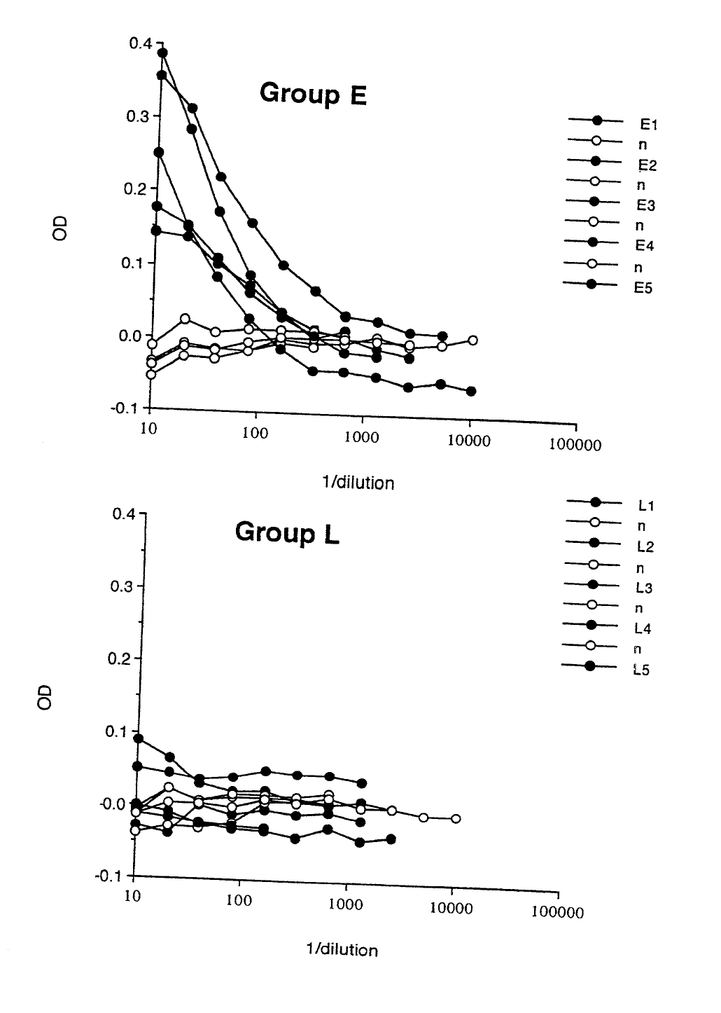

FIG. 2 shows that the increase in antibody responses induced by inclusion of anti-CD28 with the avidin immunisation is enhanced by association of avidin with the anti-CD28 antibody. Pre-incubation of biotinylated anti-CD28 with streptavidin to block the biotinylated sites and prevent subsequent binding of avidin, gives a much reduced antibody response to avidin. Mice in group E were immunised with biotinylated anti-CD28 mixed with avidin, while mice in group L (lower graph) received the same material but with a pre-incubation of anti-CD28 with streptavidin. The open circles on each graph, labelled (n) are titrations of normal mouse sera.

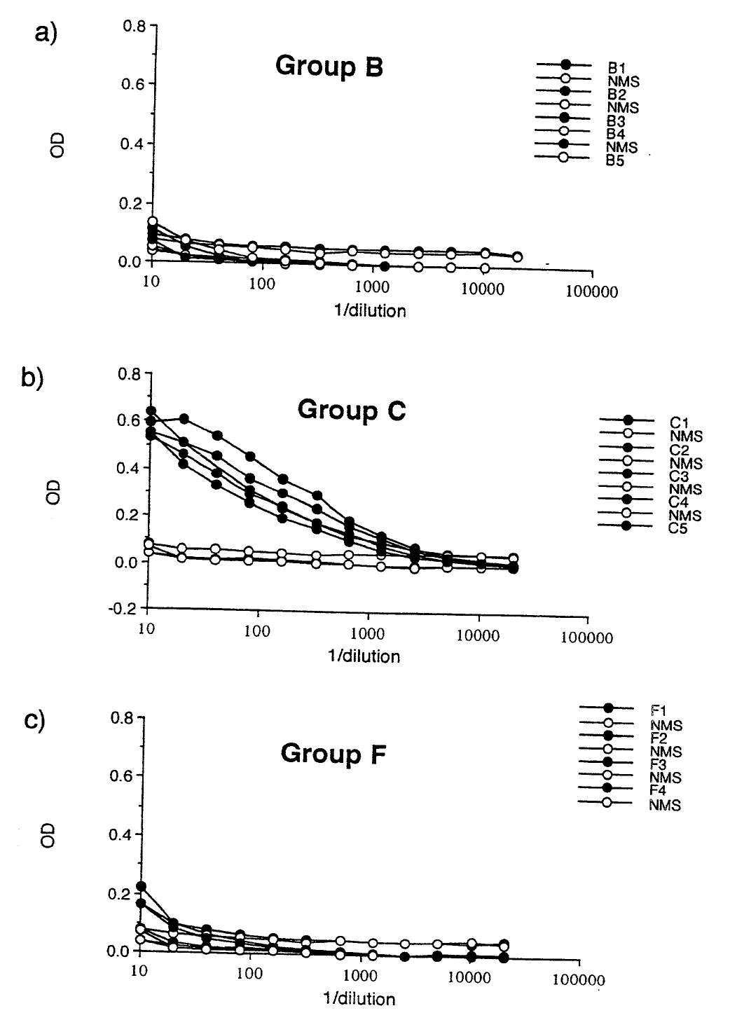

FIG. 3 again shows that the adjuvant effect of anti-CD28 is enhanced by association with the antigen, avidin. The upper graph (group B) shows the primary antibody response against avidin in response to immunisation with avidin alone. The middle graph (group C), shows the response to a single immunisation with avidin plus biotinylated anti-CD28, and the lower graph (group F) shows the response to avidin mixed with purified (un-biotinylated anti-CD28). In each case the open circles (labelled NMS) represent titrations of normal mouse sera. Un-biotinylated anti-CD28 would not be expected to associate with the antigen (avidin) as would biotin anti-CD28.

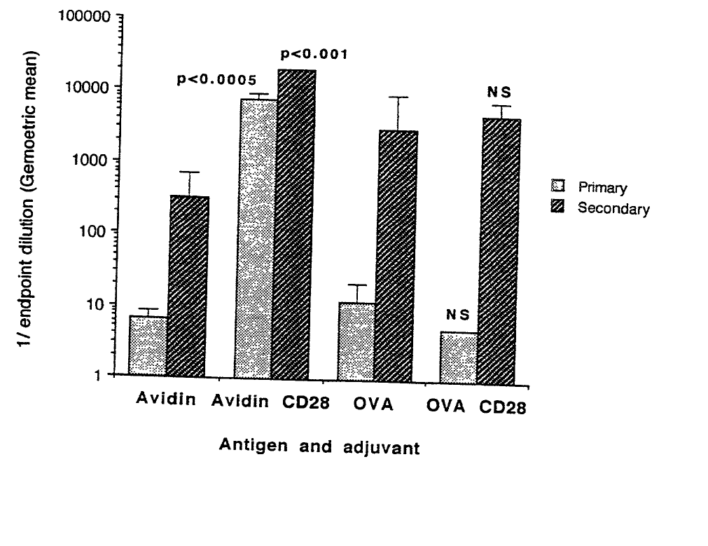

In further experiments the inclusion of ovalbumin as a negative control for the cross-linking of anti-CD28 and avidin provides convincing evidence of the requirement for a physical association between the antigen and adjuvant. There is no association between anti-CD28 and ovalbumin, consequently anti-CD28 does not enhance the antibody response to ovalbumin while very significantly enhancing the response to avidin, FIG. 4.

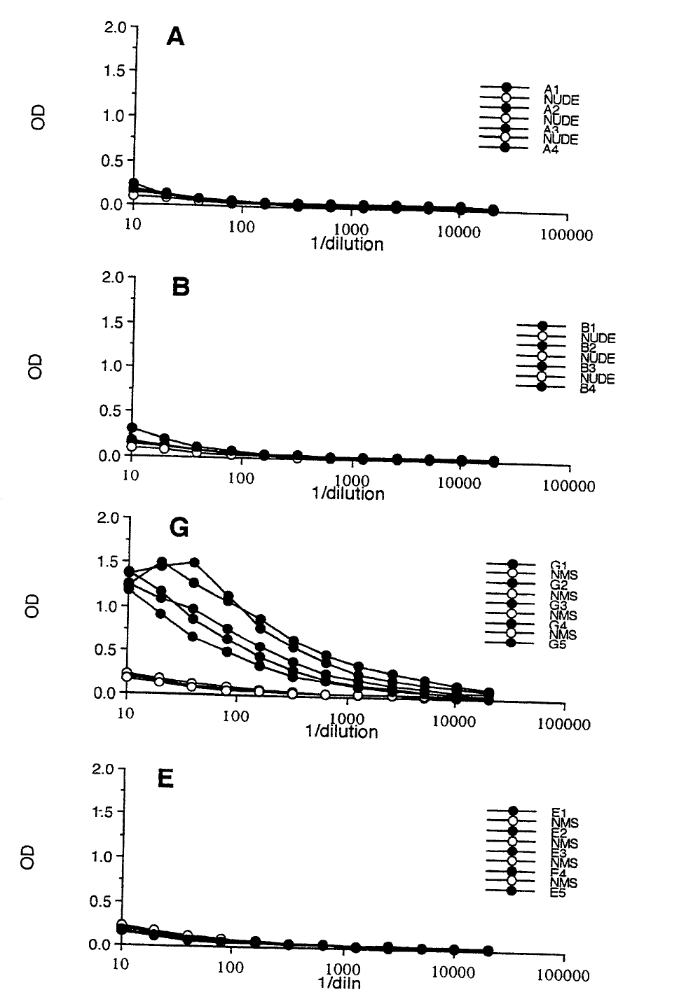

FIG. 5 shows that the adjuvant effect of anti-CD28 is not apparent in the absence of T-cells. Biotinylated anti-CD28 does not enhance the antibody response to avidin in nude (athymic) mice which lack functional T lymphocytes. Groups A and G were immunised with avidin alone, while groups B and E were immunised with avidin plus biotinylated anti-CD28. Groups A and B were athymic nude mice, while groups G and E were their normal (euthymic) littermates.

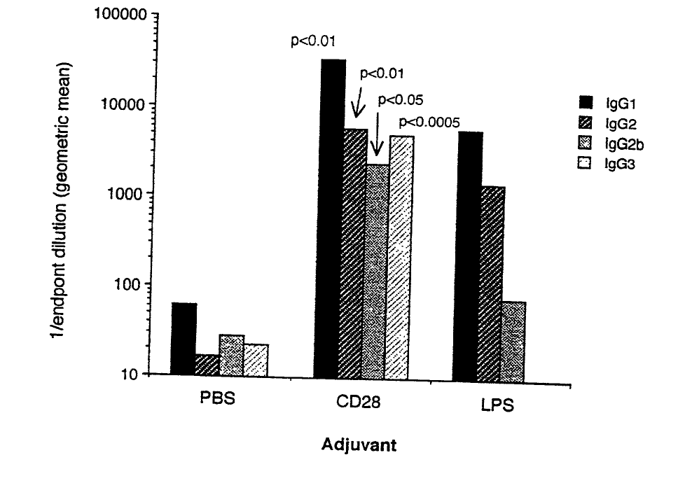

FIG. 6 shows that the antibody response to the avidin/anti-CD28 immunogen elicits the production of each of the IgG subclasses, IgG 1, IgG 2a, IgG 2b and IgG 3. This response pattern is distinct from that shown by the Salmonella typhosa lipopolysaccharide producing significantly elevated titers of IgG 2b and also the production of IgG 3.

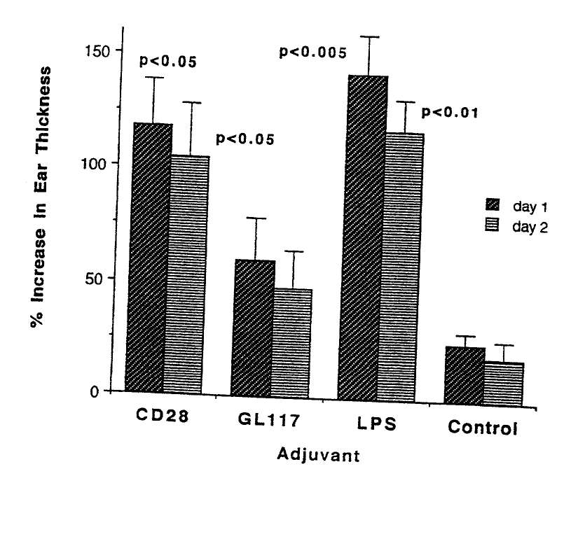

FIG. 7 shows that T-cell responses against avidin are also enhanced by biotinylated anti-CD28. This figure shows delayed type hypersensitivity (DTH) responses to an injection of 10 μg of avidin in to the ear pinna. Ear swelling in the avidin injected ear in relation to a control (PBS injected) ear is shown. Mice immunised four weeks earlier with avidin plus biotinylated anti-CD28 showed significantly higher DTH responses to avidin than mice which were immunised with avidin alone. DTH responses are thought to be mediated by the T helper 1 population.

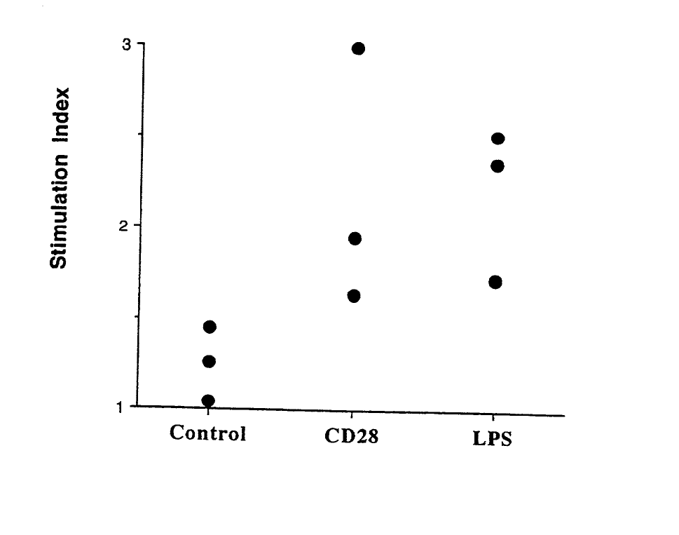

Finally, in support of this observation, FIG. 8 represents the effect of the avidin/anti-CD28 immunogen on T-lymphocyte proliferation. The avidin/anti-CD28 complex stimulates T-lymphocyte multiplication at least as effectively as SalmonelLa typhosa lipopolysaccharide and is significantly more effective at inducing proliferation than control treatment.

It is evident from the foregoing description that the murine model serves to assist in exemplifying the method of T-lymphocyte activation via the CD28 receptor. The use of the method in medical or veterinary practice may require the development of species specific antibodies to enable efficient T-lymphocyte activation within a defined species.

### Materials and Method

Antibodies and Adjuvants

Avidin and ovalbumin was purchased from Sigma (Poole, Dorset) Biotinylated and non-biotinylated anti-CD28 and biotinylated control hamster IgG were purchased from Pharmingen (Cambridge Bioscience, Cambridge UK). Gerbu adjuvant was obtained from QueMaCo Ltd, Barnstone, Notts. Salmonella Typhosa lipopolysaccharide was purchased from Sigma.

Animals and Immunisations

6-8 week old female BALB/c mice were purchased from the University of Sheffield Field Laboratories and used at 8-12 weeks of age.

To allow efficient conjugation of avidin and biotin, avidin at 1 mg/ml, and biotinylated antibody at 1 mg/ml were mixed together at a 1:1 ratio and left on ice for 30 minutes. The ovalbumin experiment was performed in exactly the same way, but with ovalbumin substituted for avidin. This incubation was done in parallel with the avidin/anti-CD28 treatment. The conjugates were then diluted in PBS to give a total of 10 μg antibody and 10 μg avidin in 0.2 ml PBS, which was then injected intraperitoneally. In cases where avidin alone was used it was pre-mixed with an equal volume of PBS and left on ice for 30 minutes before dilution and injection. Lipopolysaccharide was similarly mixed with avidin to give a final dose of 10 μg per mouse. Gerbu adjuvant was used as instructed by the manufacturer.

Measurement of Immune Responses

Mice were bled at 10 days post immunisation, and anti-avidin antibody titres assayed by ELISA as follows. ELISA plates (CoStar) were coated overnight at 4° C. with avidin at 10 μg/ml in PBS. Plates were blocked with 3% BSA in PBS for one hour at room temperature, washed with PBS-0.05% Tween 20 using an Elcatech plate washer (Elecatech, Salem, N.C., USA), and then serial dilutations (100 μl) of antisera in PBS 3% BSA were incubated on the plates for 2 hours at room temperature. Following washing the conjugate was added (Horse radish peroxidase conjugated goat anti-mouse immunoglobulins or anti-mouse isotype specific conjugates; Southern Biotechnology, from Harlan-SeraLab, Sussex) and incubated for one hour at room temperature. Following further washing the substrate was added. O-phenylenediamine dihydrochloride (Sigma) was diluted to 0.5 mg/ml in Citrate-phosphate buffer, pH5. After 20 minutes incubation at room temperature the reactions were stopped by the addition of 50 μl 1M sulphuric acid, and optical densities were read at 490 nm. Endpoint titres were taken as the reciprocal of the dilution at which the OD for the test serum intersected that of the control, normal mouse serum.

Delayed type hypersensitivity responses were assessed by measurement of differences in swelling following injection of 10 μg avidin in 10 μl PBS, or 10 μl PBS alone into the ear pinnae of mice 4 weeks after immunisation with avidin alone, avidin plus biotin anti-CD28, avidin plus biotinylated IgG, or avidin plus LPS.

T-cell proliferation was monitored using ³H-thymidine incorporation by isolated splenocytes from immunized mice in response to antigen. Briefly, spleens were disrupted and erythrocytes removed by hypotonic lysis. Cells were incubated at 2×10⁵ per well in quadruplicate in 96 well plates in the presence of the indicated concentration of avidin. After 48 hours the cells were pulsed with 0.5 μC/well of ³H-thymidine for 6 hours, then harvested using a Packard cell harvester. ³H-thymidine incorporation was determined using a β-counter.

Production of a Chimeric Recombinant Fusion Protein

Particularly in the instance where large scale production is required the vaccine of the invention is produced as recombinant proteins which are expressed in a heterologous system. We prefer to provide the adjuvant of the invention co-joined to the vaccine antigen of the invention by producing a recombinant chimeric fusion protein thus fusing together a selected vaccine antigen with at least one of a number of possible CD28 binding moieties. The CD28 binding region is either an antibody fragment or a synthetic antibody fragment which binds to CD28, alternatively selected parts of the naturally occurring ligands B7.1, B7.2, or their synthetic counterparts, may be used. Indeed, it is within the scope of the invention to provide any protein which brings about stimulation of CD28, typically through binding thereto. In producing the chimeric recombinant fusion protein the antigen and adjuvant are first selected and the cDNAs provided. Then using PCR appropriate restriction sites are inserted into the antigen and adjuvant cDNAs so as to create a precise in-frame fusion between the two respective coding sequences. Ideally, the stop codon from the N-terminal of the two coding regions is removed to allow a single mRNA to be transcribed, and also ideally, the part of the C-terminal protein coding region encoding the leader sequence and any anchor domains is also removed to prevent the chimeric protein sticking to the cell membrane. Once the hybrid cDNA is prepared it is ligated, using conventional techniques, into an appropriate expression vector, which is selected having regard to the nature of the host used to manufacture the fusion protein. Suitable hosts include bacterial cells such as cells of Escherichia coli, or alternatively an insect system in which case a baculovirus vector is chosen. If preferred, other host systems may be employed such as yeast cells or mammalian cells. We also prefer to provide the hybrid cDNA with suitable secretion signals so as to arrange for secretion of the fusion protein into the host system supernatant, since this facilitates harvesting of the vaccine.

### Results

FIG. 1 shows results from two separate experiments indicating that conjugation of anti-CD28 to avidin for the first immunisation very markedly enhances primary antibody responses to avidin in comparison to avidin alone, or to avidin conjugated to a control hamster IgG-biotin. Primary antibody responses are enhanced by anti-CD28 to a greater extent than either the commercially available adjuvant, Gerbu, or 10 μg of LPS. The lower part of the figure shows that this adjuvant effect of anti-CD28 is maintained following a boost with avidin alone in that secondary antibody responses against avidin are also significantly increased, indicating that anti-CD28 enhances immunological memory. This enhancement of secondary responses is retained several months after primary immunisation (data not shown).

The preference for physical linkage of antigen and anti-CD28 is demonstrated in FIGS. 2, 3 and 4. If biotin anti-CD28 were pre-mixed with streptavidin to compete for biotin binding, prior to addition of avidin, adjuvanticity is lost (FIG. 2 lower panel) in comparison with the direct mixture of biotin anti-CD28, and avidin (upper panel). When avidin was mixed with un-biotinylated anti-CD28, again there was a lower adjuvant effect (FIG. 3, lower panel is purified, non-biotinylated anti-CD28, middle panel is biotin anti-CD28 plus avidin, and upper panel is avidin alone). In both of these cases, where no avidin/anti-CD28 association should have taken place, the adjuvant effect of anti-CD28 was much reduced, although there may be some effect using purified anti-CD28. Moreover, the inclusion of ovalbumin, an antigen incapable of cross-linking to anti-CD28, supports the contention that a physical association has to occur to promote the adjuvant effect, FIG. 4.

The results in FIG. 5 show that anti-CD28 had no detectable adjuvant effect in nude mice which are lacking in functional T lymphocytes, while there was a pronounced adjuvant effect in euthymic litter mates, indicating that the adjuvant effect of anti-CD28 on antibody responses requires T lymphocytes, fitting with the theory that anti-CD28 is acting as a co-stimulus to T lymphocytes in vivo.

The results in FIG. 6 indicate that each of the four IgG classes are produced in response to the avidin/anti-CD28 conjugate. Significantly, IgG2b has an increased titre and IgG3 production is induced in response to avidin/anti-CD28.

FIG. 7 shows that anti-CD28 not only enhances antibody responses but also enhances delayed type hypersensitivity (DTH) responses to avidin alone, as measured by ear-swelling, again indicating that T-cell responses (in this case T helper 1 type responses) are augmented by the inclusion of biotin anti-CD28 with the avidin immunisation.

Finally, FIG. 8 supports the results presented in FIG. 7 indicating that the avidin/anti-CD28 response enhances T-cell responses. Immunisation with avidin/anti-CD28 induces significantly greater T-cell priming than immunisation with avidin alone, as measured by in vitro splenocyte proliferation in response to avidin.

Thus we provide data showing the efficacy of our novel vaccine which essentially comprises at least a part of a T-cell dependent antigen that is capable of providing a T-cell dependent immune response and also an adjuvant which basically stimulates the T-cell receptor CD28, ideally through binding thereto.

1. A vaccine suitable for enhancing T-cell dependent immunity comprising a T-cell dependant antigen, or part thereof, and an associated adjuvant which is adapted to stimulate a T-cell lymphocyte via the cell surface receptor CD28.

2. A vaccine according to claim 1 wherein said antigen is a soluble antigen.

3. A vaccine according to claim 1 or 2 wherein said antigen is a protein.

4. A vaccine according to claims 1-3 wherein at least a part of said adjuvant is adapted to bind to CD28, or a part thereof.

5. A vaccine according to claims 1-4 wherein said antigen and said adjuvant are cross-linked theretogether.

6. A vaccine according to claims 1-4 wherein said vaccine is composed of said antigen and said adjuvant which are not physically co-joined.

7. A vaccine according to claims 1-6 wherein said adjuvant is an antibody, or an effective part thereof, which is adapted to bind to said CD28.

8. A vaccine according to claim 7 wherein said antibody is monoclonal.

9. A vaccine according to claim 8 wherein said antibody is a humanised monoclonal antibody.

10. A vaccine according to claims 1-9 wherein said adjuvant is based on the natural ligands of CD28, or B7.1 or B7.2, or an active binding fragment thereof.

11. A vaccine according to claims 1-10 wherein said adjuvant is recombinantly manufactured.

12. A vaccine according to claim 11 wherein said antigen and said adjuvant comprise a recombinant fusion protein.

13. A vaccine according to claims 1-12 wherein said vaccine comprises an immunostimulating composition adapted to elicit an enhanced cytotoxic T-cell response.

14. A vaccine according to claims 1-13 wherein said vaccine comprises, liposomes, biodegradable microspheres or an emulsion of antigen and adjuvant in oil.

15. An adjuvant for enhancing an immune response to a soluble protein wherein said adjuvant comprise an agent adapted to stimulate a T-cell lymphocyte surface receptor CD28.

16. An adjuvant according to claim 15 wherein said adjuvant is adapted to bind to at least a part of said CD28.

17. An adjuvant according to claims 15 or 16 wherein said adjuvant is an antibody, or a part thereof.

18. An adjuvant according to claim 17 wherein said antibody is monoclonal.

19. An adjuvant according to claims 18 wherein said adjuvant is a humanised monoclonal antibody.

20. A method for the manufacture of a vaccine capable of eliciting a T-cell dependent immune response comprising selecting a suitable T-cell dependant antigen, or part thereof, and combining said antigen with an adjuvant wherein said adjuvant is adapted to stimulate a T-cell specific response by stimulation of a T-cell receptor CD28.

21. A method for the manufacture of a vaccine according to claim 20 wherein said antigen and/or adjuvant is/are recombinantly manufactured and are co-joined to produce a chimeric fusion protein.

22. A method according to claim 20 or 21 wherein said antigen and adjuvant are cross-linked theretogether.

23. A system for use in the production of a vaccine capable of eliciting a T-cell dependent immune response wherein said system comprises a cell expressing a selected T-cell dependant antigen, or part thereof, and also an adjuvant capable of stimulating a T-lymphocyte receptor CD28.

24. A system according to claim 23 wherein said system is adapted so that said cell secretes said antigen and/or said adjuvant and in the instance where both are secreted they are secreted either singularly or as a co-joined fusion protein.

25. An isolated DNA molecule encoding either or both said antigen and/or said adjuvant according to claims 1-19.
